(19) United States
(12) Patent Application Publication
(10) Pub. No.: US 2013/0045175 A1
(43) Pub. Date: Feb. 21, 2013

(54) Use of Skin Permeation and Retention Enhancer Compound in Cosmetic and Pharmaceutical Compositions and Cosmetic and Pharmaceutical Products Containing Said Compound

(71) Applicants: Rosa Maria Teixeira Tagae Biaggio, Sao Jose dos Campos - SP (BR); Ana Paula Pedroso De Oliveira, Sao Paulo - SP (BR); Leda Fernanda Jesus, Sao Paulo - SP (BR); Mary Sanae Nakamura, Jundiai (BR); Philip Leite Ribeiro, Sao Paulo - SP (BR)

(72) Inventors: Rosa Maria Teixeira Tagae Biaggio, Sao Jose dos Campos - SP (BR); Ana Paula Pedroso De Oliveira, Sao Paulo - SP (BR); Jean-Luc Gesztesi, Sao Paulo - SP (BR); Leda Fernanda Jesus, Sao Paulo - SP (BR); Mary Sanae Nakamura, Jundiai (BR)

(73) Assignee: NATURA COSMETICOS S.A., Itapecerica da Serra (BR)

(21) Appl. No.: 13/519,525

(22) PCT Filed: Jan. 6, 2011
(86) PCT No.: PCT/IB11/00018

§ 371 Date: Nov. 8, 2012

(30) Foreign Application Priority Data
Dec. 29, 2009 (FR) 09 59634

(51) Int. Cl. A61K 47/44; A61K 8/92; A61P 17/10; A61P 17/00; A61Q 19/00; A61Q 19/02; A61K 31/522; A61K 31/352

(52) U.S. Cl. 424/62; 514/784; 514/263.34; 514/456

(57) ABSTRACT

The present invention relates to the use of a compound of general formula: wherein X is a CiO-24 carbon chain with “n” unsaturations wherein “n” is an integer from zero to 3 for the preparation of a cosmetic composition and a pharmaceutical composition to increase the skin permeation and retention of active ingredients. The present invention is suitable for cosmetic and pharmaceutical products the activity of which is enhanced with increased permeation and retention of active ingredients in the skin as a treatment for cellulite and fat accumulation, acne treatment, skin whitening amongst others.

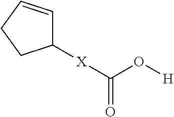

### FIELD OF INVENTION

The present invention refers to cosmetic and pharmaceutical compositions that show improved skin permeation and retention of active ingredients as well as substances that can enhance the skin permeation and retention of active ingredients.

### BACKGROUND OF THE INVENTION

Adequate skin absorption is a requirement for active cosmetics and pharmaceuticals applied topically. The skin absorption is determined not only by the partition coefficient of the active ingredient, but also by other physical-chemical characteristics thereof, particularly their solubility in water or its lipophilicity, molecular size and diffusion (Bonina et al. 1996; Saija et al. 1998).

The main obstacle to an efficient release of bioactive substances through the skin consists of the stratum corneum barrier, which prevents the active compound to reach the deeper skin layers (Bonina et al., 1995). The extent and speed with which an active ingredient penetrates the skin depends on the effect that the formulation, the skin and the active ingredient itself have in the diffusion process (Rangarajan & Zatz, 2001).

A cosmetic formulation will be effective only if the active ingredient penetrates the skin and reach the deeper layers. Besides being able to pass through the barrier of the stratum corneum, the active ingredient must reach the epidermis and dermis in a concentration adequate to perform the desired action.

The skin barrier can hinder the transdermal delivery of therapeutic agents. In this case, permeation promoters may be used in order to facilitate the delivery of that active ingredient in the skin (Williams & Barry, 2004).

Permeation promoters are compounds that penetrate the skin, reducing the resistance of the skin barrier. They should preferably be nonirritating, non-allergenic and non-toxic compounds (Williams & Barry, 2004). Numerous compounds are listed as permeation promoters, and illustrative examples are sulfoxides (such as dimethyl DMSO), Azones (such as laurocapram), pyrrolidones (such as 2-pyrrolidone), alcohol (such as ethanol), glycols (such as propylene glycol which is a usual carrier for topical formualtions), surfactants and terpenes (Williams & Barry, 2004). The permeation promoters known form prior art are predominantly of synthetic and mineral origin.

In view of this scenario, the objective of the present invention was to develop a permeation promoter of vegetal origin, such as fatty acids derived from sapucainha.

The “sapucainha” (caulmoogra or Carpotroche brasiliensis) is known for its antibacterial and anti-inflammatory properties. More recently, as revealed by the patent FR 2 706 304, sapucainha oil was disclosed as a component of cosmetic compositions for harmonization of skin pigmentation.

Still, according to patent FR 2 518 402, sapucainha oil may be useful to normalize sebaceous secretions and microbial flora of the skin.

Patent FR 2 876 908 discloses the use of chaulmoogra oil and/or its components for the preparation of a pharmaceutical or cosmetic composition for the prevention of fat accumulation and cellulite.

Patent FR 2 876 909 teaches a cosmetic and/or pharmaceutical composition for the treatment and prevention of lipogenesis and cellulite. This composition is characterized by comprising one or more xanthine bases—theobromine, caffeine and theophylline—in combination with chaulmoogra oil or at least one of its constituents—chaulmoogric acid, hydnocarpic acid and gorlic acid.

Patent FR 2 930 782 discloses a process for obtaining fatty esters from fatty acids derived from vegetable oils and butters, including the sapucainha through an enzymatic process, so that such esters can act as emollients, emulsifiers and co-emulsifiers with distinguished performance, with higher slip properties and spreadability when compared with the products known form the state of the art.

It can be concluded that no prior art document discloses the use of vegetal compounds or their derivatives as a promoter of skin permeation and retention of active ingredients in cosmetic and pharmaceutical compositions.

### SUMMARY OF THE INVENTION

The present invention refers to the use of a compound of general formula:

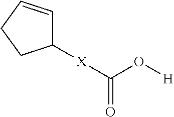

wherein X is a C₁₀₋₂₄ carbon chain with “n” unsaturations wherein “n” is an integer from zero to 3 for the preparation of a cosmetic composition and a pharmaceutical composition to increase the skin permeation and retention of active ingredients.

The present invention also refers to cosmetic and pharmaceutical compositions and products comprising the above compound up to the intended use.

### BRIEF DESCRIPTION OF THE DRAWINGS

FIG. 1 illustrates fatty acids obtained from sapucainha.

FIG. 2 illustrates a Franz cell.

FIG. 3a is a graph showing the percentage of total active ingredients applied to the skin that has permeated same throughout the study using a saturated solution of theobromine in propylene glycol containing 10% of oleic acid versus a sample control (without oleic acid). The white bar refers to a control (without oleic acid) and gray bars refer to a sample containing 10% of oleic acid.

FIG. 3b illustrates the skin retention of active theobromine after 24 hours of study: (A) Total amount of active ingredient retained in the area of skin permeation, (B) amount of retained active ingredient compared to the total active ingredient amount in the sample applied and used during that study.

FIG. 4a is a graph showing the percentage of total active ingredients applied to the skin that has permeated same throughout the study using a saturated solution of theobromine in propylene glycol containing 10% sapucainha fatty acids versus a sample control (without sapucainha fatty acid). The white bar refers to a control (without sapucainha fatty acid) and gray bars refer to a sample containing 10% of sapucainha fatty acid.

FIG. 4b illustrates the skin retention of active theobromine after 24 hours of study: (A) Total amount of active ingredient retained in the area of skin permeation, (B) amount of retained active ingredient compared to the total active ingredient amount in the sample applied and used during that study.

FIG. 5 is a graph showing the percentage of total gallic acid applied on the skin that has permeated same throughout the study. The white bars refer to a control and gray bars refer to a sample containing 5% sapucainha fatty acids.

FIG. 6 is a graph showing the percentage of total quercetin applied on the skin that has permeated same throughout the study. The gray bars refer to a sample containing 5% sapucainha fatty acids.

FIG. 7 is a graph showing the percentage of total gallic acid applied on the skin that has permeated same throughout the study. The white bars refer to a control and gray bars refer to a sample containing 5% sapucainha fatty acids.

### DETAILED DESCRIPTION OF INVENTION

The present invention relates to the use of a compound of general formula:

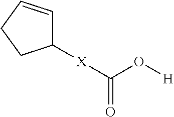

wherein X is a C₁₀₋₂₄ carbon chain with “n” unsaturations wherein “n” is an integer from zero to 3 for the preparation of a cosmetic composition to increase the skin permeation and retention of active ingredients.

The present invention also refers to cosmetic and pharmaceutical compositions and products comprising the above compound up to the intended use.

Preferably the compound used in accordance with the present invention is selected from compounds having the general formula

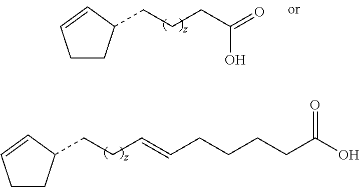

wherein z is an integer from 5 to 9, or mixtures thereof.

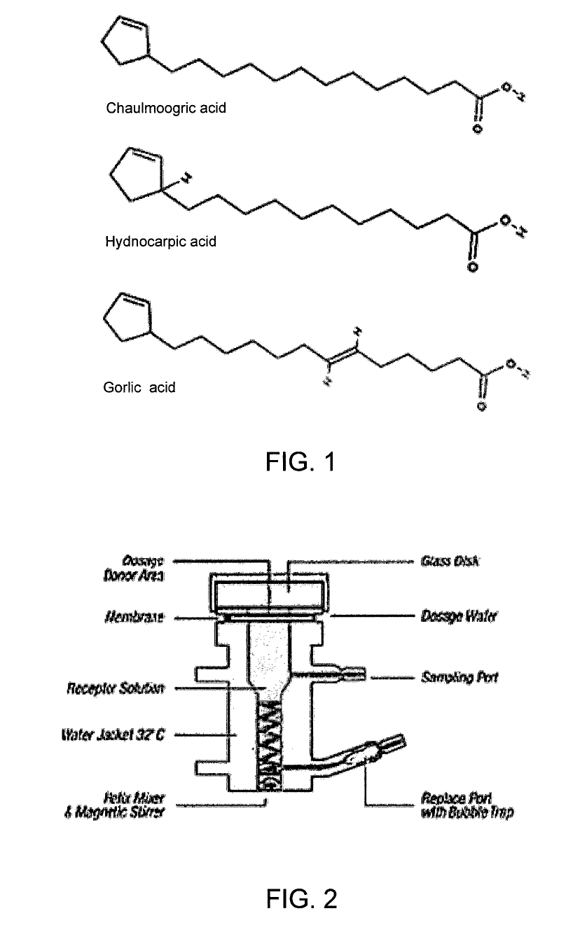

In a preferred embodiment, the compound used in the present invention is a fatty acid derived from the sapucainha and selected from chaulmoogric acid, hydnocarpic acid and gorlic acid, or mixtures thereof (FIG. 1).

The fatty acids used in accordance with this preferred embodiment are obtained from sapucainha (Carpotroche brasiliensis). Still, sapucainha may be used in the present invention in the form of oil, butter and/or an extract.

Table 1 below shows preferred physical-chemical characteristics of a sapucainha oil to be used in the present invention.

TABLE 1
Physico-chemical characterization of sapucainha oil

| Physico-chemical | Crude oil specification |
|---|---|
| Physical Aspect 25° C. | Solid |
| color | White - yellow cream |
| Odor | Typical |
| Saponification Index (mgKOH/g) | 200.0 to 220.0 |
| Iodine Index (g I2/100 g) | 12.0 to 16.0 |
| Acidity Index (mgKOH/g) | max: 10.0 |
| Peroxide Index (meg/kg) | max: 10.0 |
| Humidity (%) | max: 0.5 |
| C16:0 (palmitic) | 4.0 to 8.0 |
| C18:1 (oleic) | 1.0 to 5.0 |
| Chaulmoogric | 40.0 to 48.0 |
| Hydnocarpic | 26.0 to 34.0 |
| Gorlic | 10.0 to 20.0 |

In a preferred embodiment of the present invention, the fatty acids obtained from sapucainha are used to increase the cutaneous permeation and/or retention of active ingredients selected from the group consisting of tannins, flavonoids and/or xanthine, or combinations thereof, in cosmetic and/or pharmaceutical compositions. More preferably, the tannin used in the present invention is gallic acid, the flavonoid is quercetin and xanthine is theobromine.

The compositions containing fatty acids of sapucainha used in accordance with the present invention may be in the form of cream, ointment, gel, paste, emulsion, suspension, tincture.

The compositions according to the present invention can be used, for example, for the permeation of anti-cellulite active ingredients or in the cosmetic treatment of the face, or to the permeation of topical vaccines, for sensitive skin, acne, anti-aging and for skin whitening.

Preferably, a combination of fatty acids obtained from sapucainha is present in an amount of about 0.01 to about 20% by weight of the total composition.

Sapucainha fatty acids may also be used in combination with propylene glycol.

The following examples are preferred embodiments of cosmetic and/or pharmaceutical compositions containing the permeation and/or retention agents object of this invention but should not be interpreted as a limitation thereof. In this sense, it should be understood that the scope of the present invention encompasses other possible embodiments within the content of the claims, including the possible equivalents thereto.

TABLE 2
Example of cosmetic composition containing sapucainha
fatty acid as a permeation promoter for quercetin.

| Component Name | Concentration (% by weight) |
|---|---|
| Disodium EDTA | 0.100 |
| Water | 53.074 |
| Gum | 1.000 |
| bidistilled glycerin | 6.000 |
| hydroxyethyl acrylate, copolymer of | 1.000 |
| acryloyldimethyl | |
| Sodium hydroxide | 0.010 |
| ethyl alcohol 96 | 21.000 |
| Propylene | 12.000 |
| sapucainha fatty acid | 5.000 |
| Preservatives | 0.800 |
| Quercetin | 0.016 |

### a) Test for Permeation In Vitro—Protocol

In order to determine the best way to test the action of the permeation agents, the inventors carried out a literature review, searching for publications that disclosed evaluation of enhancer compounds. Seven articles describe a model of action to promote permeation of active ingredients. In general, they followed the protocol set forth below:
Pre-treatment of the skin—skin in contact with a solution containing enhancer (for 14 h to 24 h before the permeation study), followed by more than 24 hours of permeation.
Skins: hairless rat, rabbit ear, human cadaver and pig's ear.
Application of saturated solution containing active ingredient and the enhancer added to this solution.

Upon checking the articles and publications found in the search, the inventors followed the model described in R. Thakur A. et. al, 2007. The model uses pig skin without any pretreatment. The pig skin has characteristics very similar to the human skin thus providing more reliable results. Furthermore, they opted for a model without pre-treatment to avoid disruption of the skin, which would cause interference in the results.

Fresh pig ears were obtained at slaughterhouse immediately after killing the animal and before the carcass was subjected to the procedure of scalding. The material was washed with the aid of a sponge using only tap water at room temperature.

The ears edges were cut with dissection scissors and the skin was removed with the aid of a scalpel. The researches selected areas where the skin was intact and without any type of abnormality. These regions were dermatomized to obtain slices of skin with a maximum thickness of 0.5 mm. The skin samples were then washed in PBS (phosphate buffered saline) containing antibiotics and stored at −20° C. until they were used (durability: up to 4 weeks). On the day of the experiment, the skin samples were removed from the freezer and kept at room temperature for half an hour before starting the experiment.

The skin sample was placed in a Franz cell (as illustrated in FIG. 2) with the dermal side facing the receptor chamber and left in contact with the receptor liquid for thirty minutes to get hydrated. The experiment was conducted in infinite dose. After the hydration time, the formulation under study was applied under the skin with the aid of a repipet (Eppendorf). 300 mL of formulation were administered and the formulation was tested by means of six replicates carried out simultaneously.

After application of the formulation, the system was closed and all bubbles that might be formed were removed from the chamber. The donor chambers were sealed in order to provide a occlusive condition for percutaneous permeation. At the end of the assembly of the cells it was started the count of the permeation time.

The percutaneous permeation was determined by the amount of active ingredient capable of penetrating until the receptor liquid. To determine the percutaneous permeation the receptor fluid was collected at times 2, 4, 6, 8, 10 and 24 hours from the onset of the test. Each sample was collected by removing 1 mL of liquid present in the receptor chamber. The collected material was then subjected to HPLC analysis to quantify the content of the active ingredient.

At the end of the study the Franz cells were disassembled. The excess of product was removed from the skin samples with the aid of cotton swabs having soft cotton ends. The epidermal surface of the skin samples waswashed gently with distilled water and then dried on paper towels. The liquid in the receptor chamber was discarded. The part of the skin samples that were in contact with the formulation were cut, fragmented and placed in flasks of 50 mL. It was then added 4 mL of methanol in each flask and skin was homogenized with an Ultra-Turrax using homogenization speed of 25,000 rpm to obtain a suspension as homogeneous as possible. The ultra-Turrax rod was washed with distilled water followed by a washing with ethanol between each homogenizing step. The material was then homogenized using a vortex shaker for about 1 minute left in ultrasonic bath for 30 minutes. At the end of sonication, the supernatant was removed and subjected to quantification of the content of active ingredient.

The receptor liquid samples collected and the extraction of the skin had the contents of the active ingredients analyzed by high performance liquid chromatography (HPLC).

The determination of the cutaneous retention was carried out by measuring the content of the active contained in the skin used in the Franz cell at the end of 24 hours of study.

### b) Comparative Test: Oleic Acid X Sapucainha Fatty Acids

The objective of this test was to validate the method and evaluating the potential of sapucainha fatty acids as promoters for permeation and retention. The test was performed comparing the effects of sapucainha fatty acid with the effects of oleic acid, which is known as a promoter of permeation.

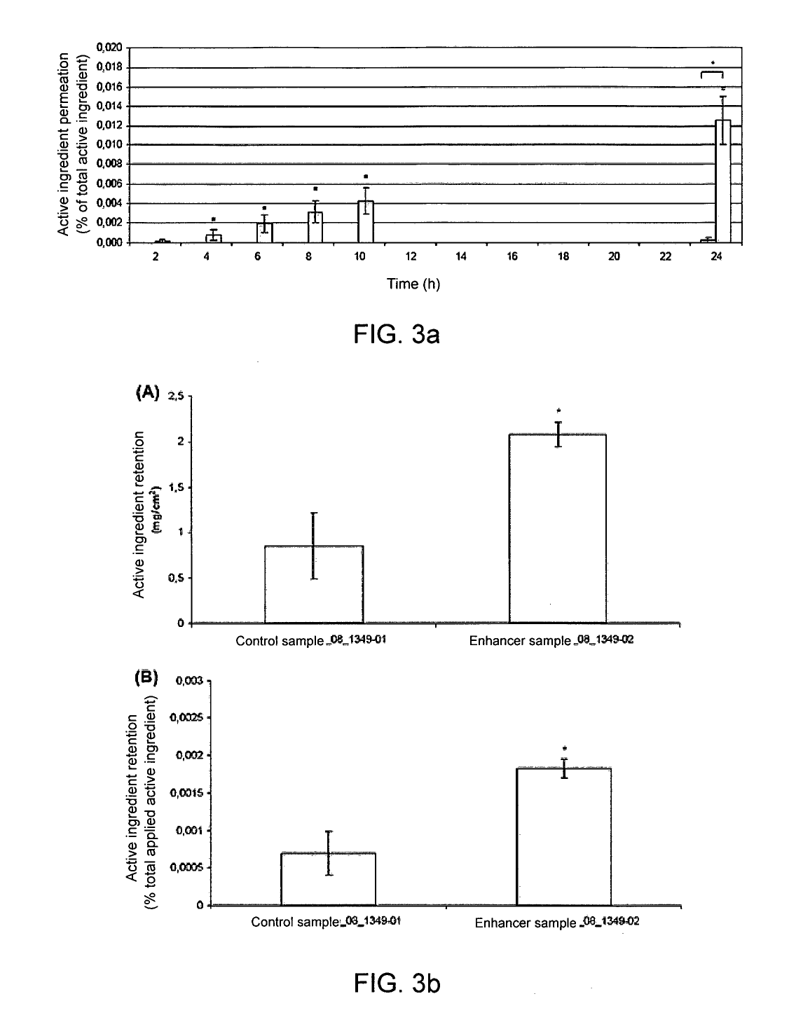

A saturated solution of theobromine was prepared (with and without oleic acid—10% solution). The solution free of oleic acid is the negative control solution and the one with oleic acid is the positive control. Theobromine was used as a marker. The permeation and retention results obtained for oleic acid are shown in Table 3 and FIGS. 3a and 3b.

TABLE 3
Results of Theobromine retention in
the skin (control and oleic acid).

| | | Retention in | Receptor |
|---|---|---|---|
| Solution | Release | the Skin | Solution |
| Negative control | infinite dosage | 0.8 ug/cm2 | tends to zero |
| Positive Control | infinite dosage | 2.0 ug/cm2 | 14 ug/cm2 |
| (10% oleic acid) | | | |

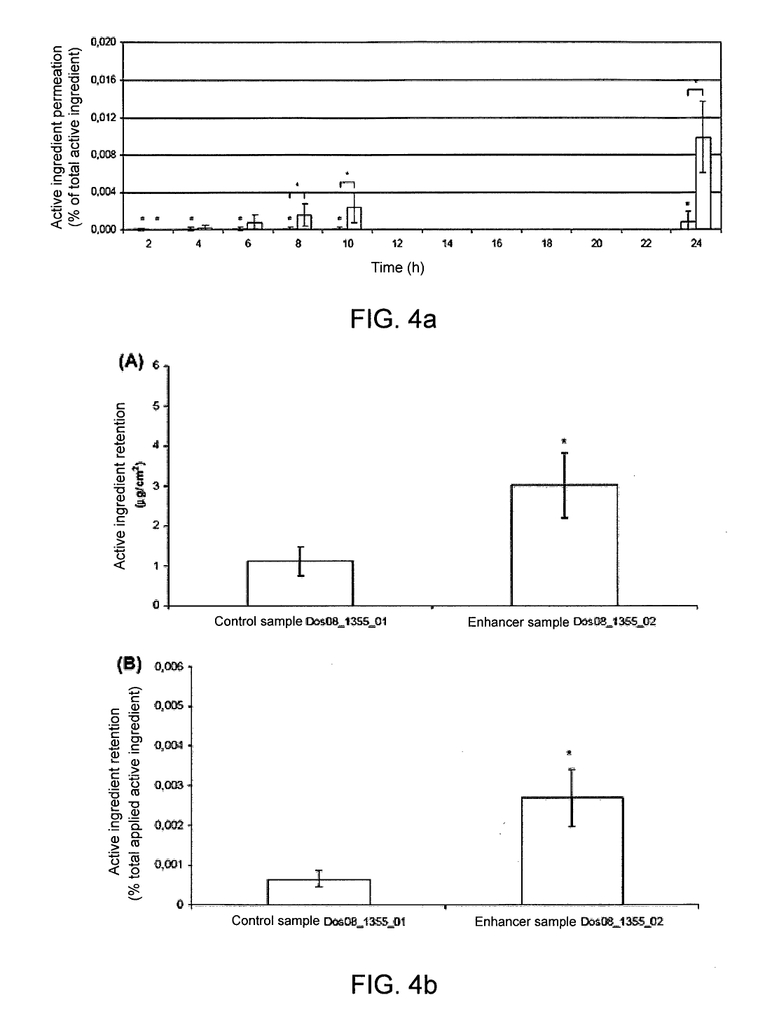

The same test was repeated, this time replacing the oleic acid with sapucainha fatty acid. The retention results are shown in Table 4 and FIGS. 4a and 4b.

TABLE 2
Results of Theobromine retention in the skin
(control and sapucainha fatty acids).

| Solution | Release | Retention Skin | Receptor Solution |
|---|---|---|---|
| Negative control | infinite dosage | 1.1 ug/cm2 | 0.6 ug/cm2 |
| Positive Control | infinite dosage | 3.0 ug/cm2 | 11.3 ug/cm2 |
| (10% sapucainha | | | |
| fatty acids) | | | |

Upon comparing the results obtained for oleic acid and sapucainha, it is observed that the permeation and retention profiles are similar and, therefore, it was concluded that the fatty acids obtained from sapucainha have promoting effect of permeation and retention of active ingredients similar to acid oleic acid on the employed model.

### c) Permeation Test of Gallic Acid in Propylene Glycol

Still using the same method described above, the researchers evaluated skin permeation of gallic acid in the presence of sapucainha fatty acids.

Propylene glycol was used as the control. The samples were prepared with 5% sapucainha fatty acids in propylene glycol.

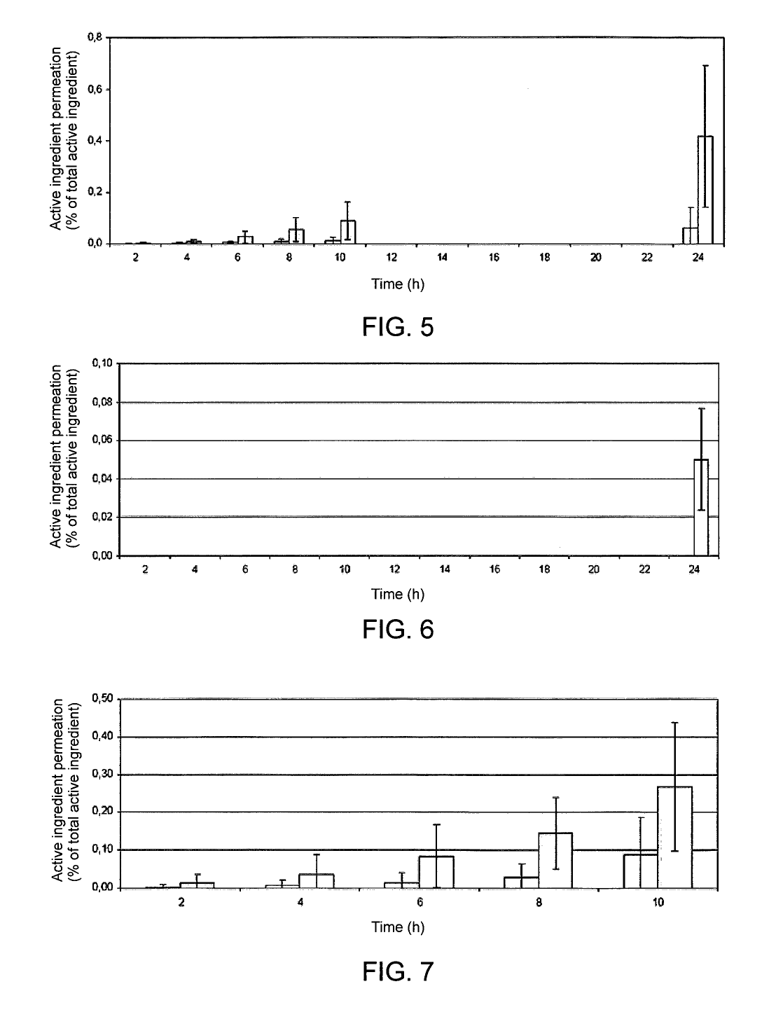

The result obtained for the percentage of active ingredient permeated in relation to the amount of active ingredient applied (FIG. 5) shows that both the sample containing 5% sapucainha fatty acids and the control assisted the permeation of the active ingredient. However, the results for the sample with sapucainha fatty acids were better, wherein 0.4±0.2% of total active ingredients was delivered after 24 hours of study for this sample and only 0.06±0.08% was delivered for the control sample.

### d) Permeation Test of Quercetin in Propilenogligol

The results show that there was no skin permeation in the control sample with quercetin (the results for the control are not even visible in the graph). As for the sample with 5% sapucainha fatty acids in propylene glycol permeation was observed after 24 hours of study. The amounts of permeated active ingredient are shown in FIG. 6. The sample with sapucainha fatty acids was able to deliver an amount of 0.05±0.02% of total active ingredients after 24 hours of study.

### e) Test Permeation of Gallic Acid in Alcohol Gel

The same study described above was conducted using alcohol gel as the control. The sample tested was prepared in an alcohol gel with 5% sapucainha fatty acids and gallic acid as a marker. The alcohol gel with sapucainha fatty acids presented the highest amount of active ingredient delivery within 10 hours of study when compared with control (FIG. 7). The alcohol gel with sapucainha fatty acid showed average permeation of active ingredients of 0.2±0.1% while the control showed only 0.08±0.09%.

### BIBLIOGRAPHIC REFERENCES

BONINA, F.; LANZA, M.; MONTENEGRO, L.; PUGLISI, C.; TOMAINO, A.; TROMBETTA, D.; CASTELLI, F.; SAIJA, A. Flavonoids as potential protective agents against photo-oxidative skin damage. Int. J. Pharm., v.145, p. 87-94, 1996.
RANGARAJAN, M.; ZATZ, J. L. Kinetics of permeation and metabolism of α-tocopherol and α-tocopheryl acetate in micro-Yucatan pig sin. J. Cosmet. Sci., v.52, p. 35-50, 2001.
WILLIAMS, A. C.; BARRY, B. Penetration enhancers. Advanced Drug Delivery Reviews, v.56, p. 603-618, 2004.
THAKUR R. A. et. al; Transdermal and Buccal Delivery of Methylxantines through Human Tissue In Vitro. Drug Development and Industrial Pharmacy, v. 33, p. 513-521, 2007.

1. A method of treating a subject's skin, comprising: applying a composition comprising a compound of general formula: 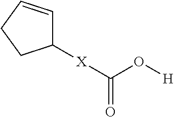
wherein X is a C₁₀₋₂₄ carbon chain with “n” unsaturations wherein “n” is an integer from zero to 3 to the subject's skin; wherein said composition increases skin permeation and retention of active ingredients.

2. The method according to claim 1, wherein the compound is selected from compounds having the general formula 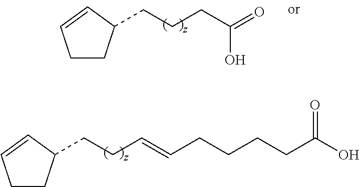
wherein z is an integer from 5 to 9, or mixtures thereof.

3. The method according to claim 1 wherein the compound is selected from chaulmoogric acid, hydnocarpic acid, gorlic acid, or mixtures thereof.

4. The method according to claim 3, wherein the chaulmoogric acid, hydnocarpic acid or gorlic acid are sapucainha derivatives.

5. The method according to claim 4, wherein the sapucainha derivatives are obtained from sapucainha oil, butter or an extract.

6. The method according to claim 1, wherein the active ingredient is selected from the group comprising tannins, flavonoids, xanthine, or combinations thereof,

7. The method according to claim 6, wherein the tannin is gallic acid, the flavonoid is quercetin and xanthine is theobromine.

8. The method according to claim 4, wherein the sapucainha derivatives are used in an amount of 0.01 to 20% by weight of the total cosmetic of pharmaceutical composition.

9. Cosmetic or pharmaceutical composition comprising a compound of 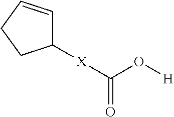
wherein X is a C₁₀₋₂₄ carbon chain with “n” unsaturations wherein “n” is an integer from zero to 3 and an active ingredient, wherein said compound enhances the cutaneous permeation and retention of said active ingredient.

10. Cosmetic or pharmaceutical composition according to claim 9, wherein the compound is selected from compounds having the general formula 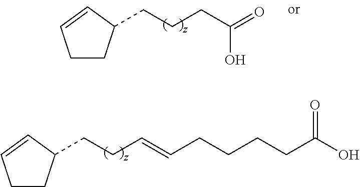
wherein z is an integer from 5 to 9, or mixtures thereof.

11. Cosmetic or pharmaceutical composition according to claim 9 for use in the treatment of cellulite, acne or for skin whitening.

12. Cosmetic or pharmaceutical composition according to claim 9 wherein the compound is selected from chaulmoogric acid, hydnocarpic acid, gorlic acid, or mixtures thereof, obtained from sapucainha and wherein said compound is present in an amount of 0.01 to 20% by weight of the total cosmetic of pharmaceutical composition.
